(19) United States
(12) Patent Application Publication
(10) Pub. No.: US 2023/0159981 A1
(43) Pub. Date: May 25, 2023

(54) DEVICE AND METHOD FOR CONCENTRATING LIQUIDS

(71) Applicant: Guild Associates Inc., Dublin, OH (US)

(72) Inventors: Ian Fleming, Hilliard, OH (US); Scott V. Angus, Worthington, OH (US); Ada S. Cowan, Delaware, OH (US)

(21) Appl. No.: 17/992,790

(22) Filed: Nov. 22, 2022

Related U.S. Application Data
(60) Provisional application No. 63/282,162, filed on Nov. 22, 2021.

(51) Int. Cl. C12Q 1/68; C12N 15/10

(52) U.S. Cl. CPC C12Q 1/68; C12N 15/1003

(57) ABSTRACT

A device for concentrating and preserving samples for subsequent analysis, wherein the samples include but are not limited to, biological samples, environmental samples, industrial samples, and the like. The present disclosure further includes a method for utilizing the device for containment, concentration, and preservation of the sample until analysis can take place. The disclosed device is simple to operate and can function without auxiliary equipment and/or sources of power making it particularly suitable for use in locations distanced and isolated from analytical facilities.

### CROSS-REFERENCES TO RELATED APPLICATIONS

This application claims the benefit of U.S. Provisional Patent Application No. 63/282,162, filed Nov. 22, 2021, and entitled DEVICE AND METHOD FOR CONCENTRTING LIQUIDS which is incorporated herein by reference.

### GOVERNMENT LICENSE RIGHTS

This invention was made with government support under contracts W81XWH-18-C-0055 and W81XWH-19-C-0055 awarded by the Department of Defense. The government has certain rights in the invention.

### TECHNICAL FIELD

The present disclosure relates to a device for concentrating and preserving samples for subsequent analysis, the samples including, but not limited to, biological samples, environmental samples, industrial samples, and the like. The present disclosure further includes a method for utilizing the device for containment, concentration, and preservation of the sample until analysis can take place. The device disclosed is simple to operate and can function without auxiliary equipment and/or sources of power making it particularly suitable for use in locations distanced and isolated from analytical facilities.

### BACKGROUND

Analytical methods developed to analyze a wide range of materials including, but not limited to, biological samples, and samples from our environment and surroundings are frequently limited by the concentration and stability of the component being analyzed. For example, an accurate diagnosis of infectious diseases is essential in providing appropriate and timely treatment of infections. Biological specimens, most commonly urine and serum, frequently require cold storage between collection and analysis to preserve the integrity of antibodies and antigens and to lessen the risk of bacterial growth and sample acidification. In some environments, such as for example far-forward resource-limited military environments and wilderness regions, such storage conditions are often not possible and failure to stabilize samples can lead to false-negative diagnostic test results. Thus, to provide high confidence diagnostic results, it is important to: (a) provide samples for analysis having sufficient analyte concentrations, and (b) avoid sample degradation. And it is particularly important to be able to achieve these objectives even when sample collection occurs in remote locations with minimal equipment and resources. The disclosure that follows addresses and provides a solution to these needs.

### SUMMARY

The current disclosure involves a device for containing, concentrating, and stabilizing a sample for future analysis without the need of manual intervention, additional equipment, and/or a power source. A first aspect of the current disclosure includes a device having first and second reservoirs, e.g., an active reservoir, and a sample reservoir, respectively, wherein each reservoir communicates with the other reservoir through a permeable membrane covering an orifice therebetween. The sample reservoir is configured to accept the initial sample and ultimately provide a concentrated sample for testing, whereas the active reservoir is configured to include a water binding agent or material and accept water removed from the sample placed in the sample reservoir.

A further aspect of this disclosure includes a sample reservoir and an active reservoir. The active reservoir includes at least one orifice and a permeable membrane covering the at least one orifice and contains at least one water binding material therein, whereas the sample reservoir includes at least one orifice and is configured to receive an aqueous sample, to removably receive the active reservoir therein, and to provide contact between the aqueous sample and the permeable membrane. The active reservoir is configured to receive, through the permeable membrane, an aqueous portion of the sample from the sample reservoir, retain at least some of the aqueous portion received by the water binding material, and provide a concentrated sample in the sample reservoir. More rapid concentration can be achieved with devices having active reservoirs with larger orifice/membrane surface areas and/or having active reservoirs with multiple orifice/membrane combinations.

A still further aspect of the current disclosure includes a device having:

(a) an active reservoir (i) including a first end and a first orifice covered with a water permeable membrane, and (ii) containing a water binding agent or material, and

(b) a sample reservoir including a second orifice and a floor opposite the second orifice.

The second orifice is for receiving and positioning the first end of the active reservoir above the floor of the sample reservoir to determine a level of concentration for a sample as water is withdrawn from the sample reservoir through the water permeable membrane and into the active reservoir. The first orifice can be located on the active reservoir's first end or a side of the active reservoir, provided the permeable membrane covered orifice can be in contact with liquid placed in the sample reservoir. The sample reservoir can include a removable (and replaceable) cap that facilitates assembly of the device and the addition of a sample to the sample reservoir. Similarly, the active reservoir can include a cap that closes the device when the active reservoir is inserted into the sample reservoir. Any cap utilized in the concentration process covers the second orifice at the upper region of the sample reservoir. The presence of the water-binding material in the active reservoir facilitates the transfer of water from the sample reservoir to the active reservoir through the water permeable membrane.

Some embodiments further include an adjusting mechanism in communication with the active and sample reservoirs configured to control a depth of insertion for the active reservoir into the sample reservoir. Examples of suitable control mechanisms include, but are not limited to, a threaded contact region between the active and samples reservoirs through the second orifice, a frictional contact region, stepped adjustment holes, and a threaded flange. An alternative to mechanisms to adjust the depth of insertion include supplying an “optional sample reservoir” that mirrors the shape of the active reservoir more closely than the sample reservoir. The optional sample reservoir could replace the sample reservoir, be nested inside the sample reservoir, or use a lip to rest on top of the sample reservoir's wall. A practical example would be a device designed to concentrate 50 mL samples to 5 mL or 10×. In some embodiments, this same device could include an optional sample reservoir that would more closely mirror the active reservoir shape allowing for concentration of a 5 mL sample to 0.5 mL. The inclusion of a water-bindable material in the active reservoir enables concentration and increasing the amount of water bindable material increases the rate and extent of concentration. Suitable water-binding materials include, but are not limited to hydrogels, molecular sieves, cellulose absorbents, super absorbent polymers, water absorbing desiccants, salts, solutes, and combinations thereof. For some embodiments including a cap, a further orifice is included in the cap and can additionally serve as a sample collection port and/or a component of the control mechanism for containing the active reservoir and for determining the insertion depth of the active reservoir. Samples can alternatively be added to the sample reservoir by removing the cap.

Depending on the nature of the samples being concentrated and transported for analysis, the sample reservoir can include a preservative configured to preserve and/or stabilize any biological or otherwise unstable materials added thereto. The active and/or sample reservoirs can be constructed from polymeric materials, metallic materials, and combinations thereof. Preferred materials of construction include transparent or clear polymeric materials typically referred to as “plastics.” In some embodiments, the active reservoir can be constructed mostly or entirely of permeable membrane. Markings placed on the circumference of the active reservoir can designate the insertion depth for the active reservoir and ultimately the final volume of the sample remaining in the sample reservoir when concentration has finished.

A further aspect of the current disclosure includes a device comprising active and sample reservoirs, where the active reservoir includes a first end and one or a plurality of water permeable membrane covering one or a plurality of orifices at the first end or on one or more sides of the active reservoir and containing a water binding material. The sample reservoir includes an orifice for inserting or receiving the active reservoir and a floor opposite the orifice. The first end of the active reservoir including the water permeable membrane is configured to be inserted into the sample reservoir to an insertion depth creating a volume within the sample reservoir between and beyond the water permeable membrane and the floor of the sample reservoir.

A still further aspect of the current disclosure includes a device having the active reservoir inserted at a fixed depth into the sample reservoir to provide a predetermined level of concentration. This configuration is particularly useful for samples requiring the same level of concentration.

Still further aspects of the current disclosure involve additional embodiments of Applicant's device for concentrating a range of samples. Such further devices include both a sample reservoir and an active reservoir, where the sample reservoir is designed to receive both an aqueous sample for concentration and an active reservoir constructed from a permeable membrane and having a water binding material therein. The two reservoirs are sized and shaped to allow the active reservoir to contact the sample contained in the sample reservoir and to provide for the removal of a predetermined volume of the aqueous sample once the active reservoir is filled and expanded to its maximum volume. The predetermined volume the sample removed can correspond to the fully expanded volume of the active reservoir, provided sufficient water binding material or agent is added to the active reservoir.

A further variation of the above device includes an active reservoir constructed from a permeable membrane and included within a rigid caged structure having openwork and a predetermined volume. The caged structure's internal volume limits the total expansion of the active reservoir constructed from a permeable membrane, thus controlling the effective predetermined volume of the active reservoir.

Still further additional aspects of the current disclosure include a device for concentrating liquids having a sample reservoir and an active reservoir configured to provide communication therebetween through a permeable membrane when the active reservoir is configured to be removably inserted into the sample reservoir. The active reservoir includes a membrane, a water binding material therein and the sample reservoir is configured to:
contain an aqueous sample in contact with the membrane of an active reservoir removably inserted therein,
provide passage of water through the membrane,
cause the water to bind to the water binding material or agent and
provide a concentrated sample in the sample reservoir.

Preferred membranes are permeable membranes.

A still further aspect of the current disclosure includes a kit for concentrating an aqueous sample. The kit includes at least the following elements:
a sample reservoir,
an active reservoir including an orifice covered by a permeable membrane, wherein the active reservoir is configured for insertion into the sample reservoir, and
a water binding material included or for inclusion within the active reservoir
   wherein the sample reservoir is configured to receive a sample for concentration, and
   wherein the active reservoir is configured to be removably inserted into the sample reservoir.

Any of the devices described above can be used alone or in combination with an integrated testing system. Such integrated systems may include a lateral flow system, a dipstick, ELISA, biological reporters, culture, and molecular methods (PCR, sequencing etc.). Such integration may require modifications of size, shape, and choice of designs to optimize this additional performance, but is well within the ability of one skilled in the art.

A further aspect of the present disclosure involves a method for concentrating a liquid utilizing the various devices described in this application with minimal variations in the procedures. A first embodiment of the method comprises the steps of:

(a) adding a sample to the sample reservoir of the device of claim 1;

(b) assembling the device of claim 1, wherein assembling the device of claim 1 provides sample contact with the membrane of the active reservoir;

(c) allowing the sample to equilibrate between the active and sample reservoirs until a desired measure of concentration has been achieved.

A still further aspect of the present disclosure involves a method including:

(a) adding a sample to be concentrated to the device's sample reservoir;

(b) inserting the device's active reservoir containing the water-binding material or agent into the sample reservoir to a depth sufficient to provide a desired measure of concentration; and

(c) allowing the liquid to equilibrate between the two reservoirs until the sample level has diminished to the lowest level of the permeable membrane or the active reservoir has reached its maximum volume, indicating that the desired measure of concentration has been achieved.

A still further aspect of the present disclosure involves a method of preserving a concentrated sample until it can be analyzed. Prior to or after the concentration, a preservative may be added to the sample reservoir.

These methods can be utilized to concentrate and preserve a range of biological liquids including, but not limited to, urine, serum, blood, cerebrospinal fluid, saliva, pleural fluid, peritoneal fluid, and amniotic fluid. The method disclosed can also be utilized to concentrate and preserve environmental and industrial liquids including, but not limited to natural water, wastewater, produced water, rain-water, and agricultural runoff. The preservative utilized can be selected based on the material included in the sample in need of preserving. For example, when biological samples are being concentrated and transported it may be important to prevent degradation of target materials and prevent bacterial or other microbial growth. Examples of preservatives found useful in preserving biological samples include, but are not limited to of osmolytes, polyols, sugars, polymers, amino acids, anionic compounds, cationic compounds, surfactants, biocides, organic solvents, and derivatives of thereof. For sampling waters containing industrial or agricultural components, preservatives can include, but are not limited to, antioxidants, polymerization inhibitors, and biocides to prevent bacterial growth at the expense of the target materials.

The time required for the fluid to equilibrate between the two reservoirs to provide the desired concentration varies depending on the nature of the sample, the nature of the membrane, the membrane's surface area, the amount of water binding material used. For a majority of liquids concentrated, it is necessary to allow the liquid to equilibrate for at least about 5 minutes, at least about 30 minutes, at least about 60 minutes, and at least about 90 minutes to provide the desired concentration level. For preferred embodiments, once the desired level of concentration has been accomplished, the active reservoir is removed from the sample reservoir, the sample reservoir is capped and transported to a testing site, where a portion of the retained sample is removed and tested. Alternatively, the entire sample may be removed and tested, or the test may be conducted in the device (example: dipstick).

The devices disclosed can be operated in a reverse manner. For example, the water binding material can be placed in the reservoir identified as the sample reservoir, and the sample placed in the reservoir identified as the active reservoir. When operated in this manner, the concentrated sample is found in the reservoir identified as the active reservoir. Some other changes to the configuration may be required to operate properly in this manner such as the permeable membrane orientation.

As noted above, any of the devices disclosed herein can be used as stand-alone systems or integrated into an existing or new testing system. Depending on the testing system utilized, some modification in size, shape or materials of construction may be required, but such modifications would be well within the parameters of the present disclosure and the ability of one skilled in the art.

### BRIEF DESCRIPTION OF THE FIGURES

FIG. 1 provides a frontal view of an embodiment of the disclosed device for concentrating small samples of liquid.

FIG. 2 provides a frontal view of an embodiment of the disclosed device for concentrating large samples of a liquid and illustrates an adjusting mechanism that determines the final volume of the concentrate.

FIG. 3A illustrates the embodiment of the disclosed device illustrated in FIG. 1 containing a liquid sample for concentration.

FIG. 3B illustrates the embodiment of the disclosed device illustrated in FIG. 1 containing a liquid sample after concentration.

FIG. 4A illustrates the embodiment of the disclosed device illustrated in FIG. 2 containing a liquid sample for concentration where the adjusting mechanism is set to provide 3 mL of a concentrate.

FIG. 4B illustrates the embodiment of the disclosed device illustrated in FIG. 2 containing a liquid sample after concentration where the adjusting mechanism has provided 3 mL of a concentrate.

FIG. 5A illustrates the embodiment of the disclosed device illustrated in FIG. 2 containing a liquid sample for concentration where the adjusting mechanism is set to provide 6 mL of a concentrate.

FIG. 5B illustrates the embodiment of the disclosed device illustrated in FIG. 2 containing a liquid sample after concentration where the adjusting mechanism has provided 6 mL of a concentrate.

FIG. 6A illustrates a Prior Art Urine cup for sampling.

FIG. 6B illustrates an embodiment of the disclosed device configured for the collection of a sample and its concentration, the device pre-set for a final volume and containing a sample. In this embodiment, the cap and the active reservoir are integrated as a single component.

FIG. 6C illustrates the embodiment of the disclosed device illustrated in FIG. 6B containing a concentrated sample.

FIG. 7 illustrates the embodiment of the disclosed device having a permeable membrane covered orifice on a side of the active reservoir.

FIG. 8 illustrates an embodiment of an active reservoir having a fixed volume to control the predetermined volume of water removed from a sample utilizing an orifice/valve combination with the valve closed when the reservoir is filled.

FIG. 9 illustrates an embodiment of an active reservoir constructed from a permeable membrane that can utilize its internal volume to control the predetermined amount of water removed from the sample and that can be used alone or within a rigid caged structure which controls the amount of water removed from the sample.

FIG. 10 illustrates the effect of temperature on the rate of concentration of a urine sample containing polyclonal antibodies.

FIG. 11 illustrates the effect of temperature on the rate of concentration of a sample of human serum containing polyclonal antibodies.

FIG. 12 illustrates the concentration of a urine sample spiked with anti-Shigella antibody PA1-7245 carried out at 22° C. for 24 hours demonstrating a relationship between the volume reduction and the absorbance increase at 450 nm.

FIG. 13 illustrates the concentration of a solution of the anti-Shigella antibody PA1-7245 in human serum over a period of 24 hours.

FIG. 14 illustrates the concentration of a solution of the anti-Shigella antibody PA1-7245 in milk over a period of 24 hours.

FIG. 15A illustrates a frontal view of an active reservoir having an orifice covered with a permeable membrane across a diagonal surface and held in place with a membrane sealer.

FIG. 15B illustrates a side view of an active reservoir having an orifice covered with a permeable membrane across a diagonal surface and held in place with a membrane sealer.

FIG. 16 illustrates a side view of a sample reservoir suitable for receiving (a) a sample and the active reservoir illustrated in FIG. 15A and 15B or (b) a sample, and the active reservoir illustrated in FIG. 15A and 15B inserted into the optional sample reservoir illustrated in FIG. 17.

FIG. 17 illustrates a side view of an optional sample reservoir suitable for receiving samples having smaller volumes and for receiving the active reservoir illustrated in FIG. 15A and 15B.

FIG. 18A illustrates a frontal view of a component of the active reservoir prior to installation of the membrane and membrane sealer, the component of the active reservoir having a plurality of orifices positioned on the two sides and the bottom of the reservoir (the latter not shown).

FIG. 18B, illustrates a permeable membrane suitable for covering the orifices in FIG. 18A.

FIG. 18C illustrates a membrane sealer for holding a permeable membrane in place on the active reservoir illustrated in FIG. 18A.

FIG. 19 provides (a) a frontal view of the components of a device before full assembly including an assembled active reservoir derived from the components illustrated in FIGS. 18A, 18B, and 18C and (b) a sample reservoir.

FIG. 20 illustrates an active reservoir including a plurality of orifices positioned on the two sides and bottom of the active reservoir (without the installation of a membrane), assembled within a sample reservoir.

FIG. 21A illustrates a frontal view of an active reservoir and a sample reservoir where the active reservoir includes at its bottom an orifice, a membrane and membrane sealer positioned at its bottom and the active reservoir is being inserted into a sample reservoir for receiving a sample and the active reservoir.

FIG. 21B illustrates a cross-sectional view of an assembled device for concentrating liquids derived from the components provided in FIGS. 21A and further having a water binding or retaining material positioned in the active reservoir where the device is in the process of concentrating an aqueous sample. The active reservoir further contains some water removed and the sample reservoir contains a portion of partially concentrated material.

FIG. 21C illustrates a view of the bottom of the active reservoir illustrated in FIG. 21A showing the membrane and the membrane sealer.

### DESCRIPTION

Applicant has developed a device for containing, concentrating, and stabilizing samples including, but not limited to biological and/or environmental samples, for later analysis without the need of additional equipment, refrigeration, and/or a power source. Applicant has also developed a kit including the device components and a method for the device's use.

The central idea of the invention involves a device capable of selectively drawing water (and in some instances other selected components included in an aqueous sample) through a permeable membrane to produce a sample having predetermined or controlled level of concentration of a component to be analysed without the need of auxiliary equipment. The concentration can be completed within a relatively short time without auxiliary equipment. The device is inexpensive to construct and simple to manufacture and/or operate. In a battlefield environment, equipment for concentrating and analysing biological samples is frequently not available, nor can samples be preserved by refrigeration. A similar situation exists when environmental samples are collected in isolated localities that lack refrigeration and/or analytical facilities. Simply adding the sample to the device prepares it for a predetermined level of sample concentration and stabilizes and/or preserves the sample without refrigeration until it can reach the necessary analytical facilities in another location up to several days or weeks later. Some embodiments provide for a variable concentration level while other embodiments are constructed to provide a fixed and predetermined concentration level. Preferred embodiments are constructed from materials that can be recycled.

Preferred embodiments of the device include an active reservoir for collecting water from a sample (and any other selected components) and holding a water-binding material in communication with a permeable membrane, wherein the active reservoir is in communication with a sample reservoir through the permeable membrane and wherein the sample reservoir optionally contains a stabilizer. The sample reservoir has a volume sufficient to hold a sample to be concentrated and the active reservoir has a volume sufficient to collect and maintain water (and any desired additional components) removed through the permeable membrane from the sample being concentrated. For some embodiments, the level of concentration can be controlled by how far the permeable membrane and active reservoir are inserted into the sample maintained in the sample reservoir. For other embodiments, the level of concentration is controlled by how much sample is placed in a device having a fixed placement of the active reservoir and permeable membrane within the cavity of the sample reservoir. For other embodiments, the level of concentration is controlled by how much water binding material is placed in the active reservoir regardless of how the placement of the active reservoir and permeable membrane within the cavity of the sample reservoir.

The rate of water passage through the permeable membrane can be affected by the choice and amount of the water binding material or agent on the permeable membrane's water-collecting side, and the choice of the permeable membrane. When the water-binding material utilized was a polysodium acrylate hydrogel (PSA), the rate of concentration (flux rate) was faster with smaller particles of PSA or higher amount of PSA up to a certain point in the active reservoir.

Passage of water and/or additional selected components across the permeable membrane is halted when the desired level of concentration has been achieved. Depending on the application, permeable membranes can be selected that allow only water to pass through the membrane or allow water and selected components to pass through the membrane based on the selected component's molecular weight, chemical structure, and the like.

In order to provide a better understanding of Applicant's invention and methods for its use, specific devices will be discussed with regard to the figures provided. The specific devices discussed below are provided to illustrate various embodiments of the disclosed invention and are not intended to otherwise limit Applicant's invention beyond the claims provided.

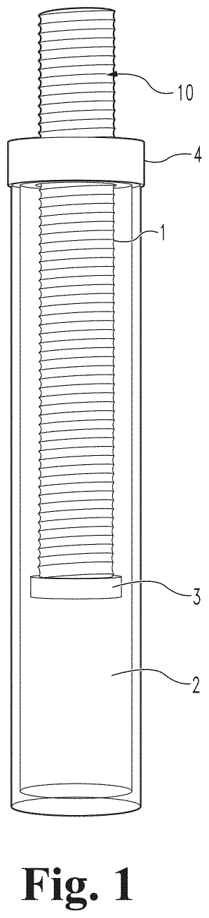

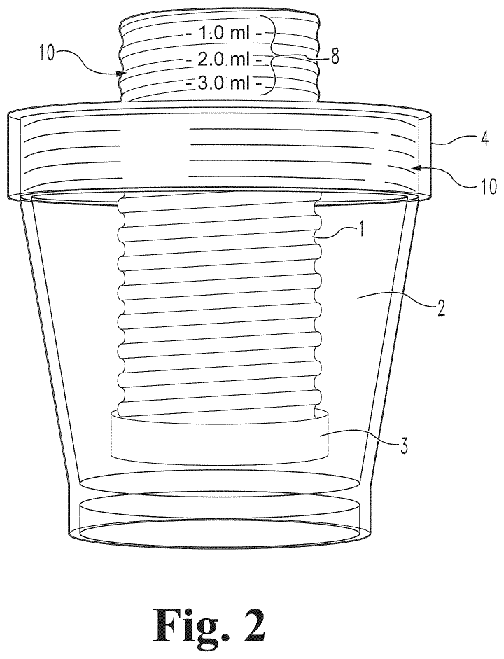

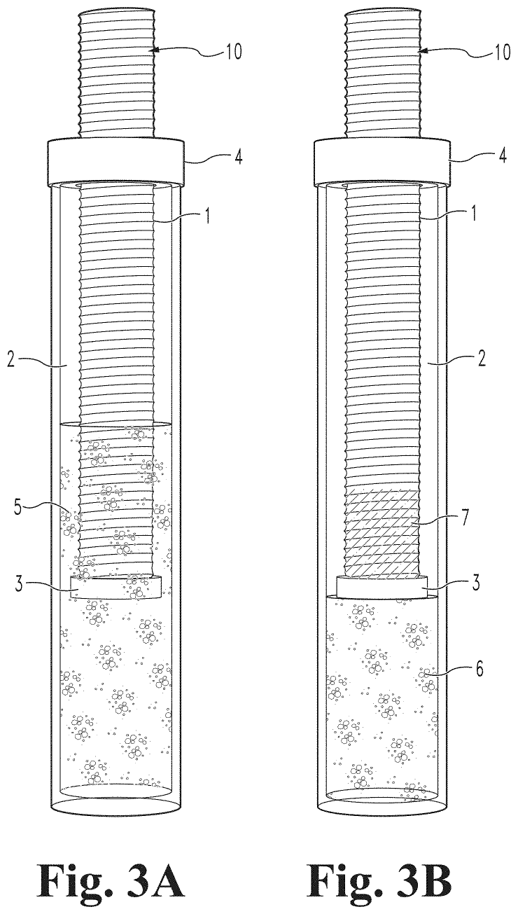

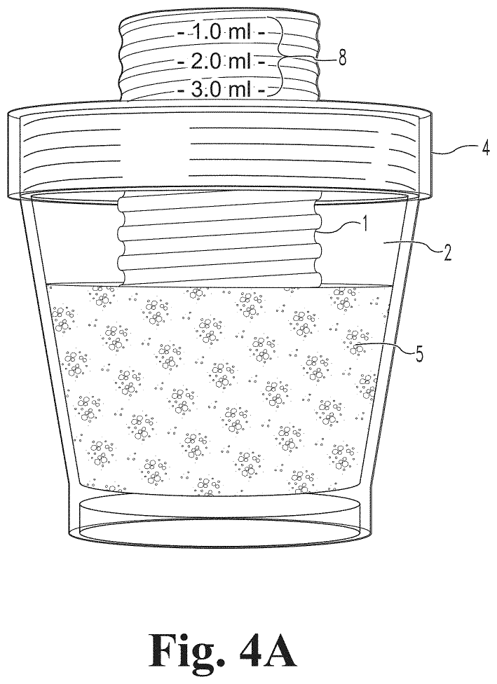

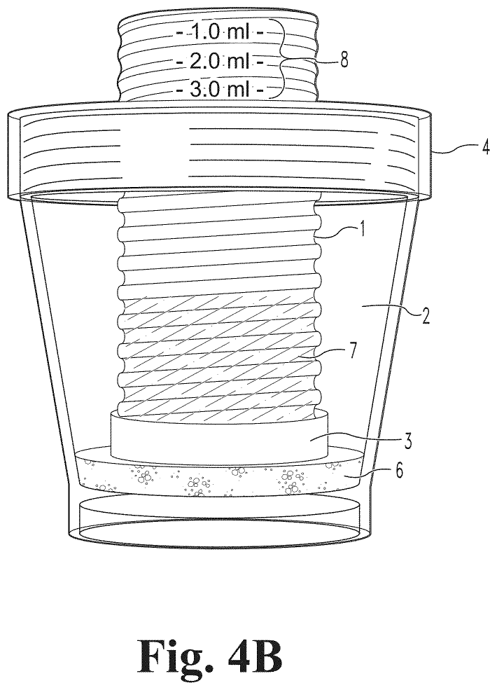

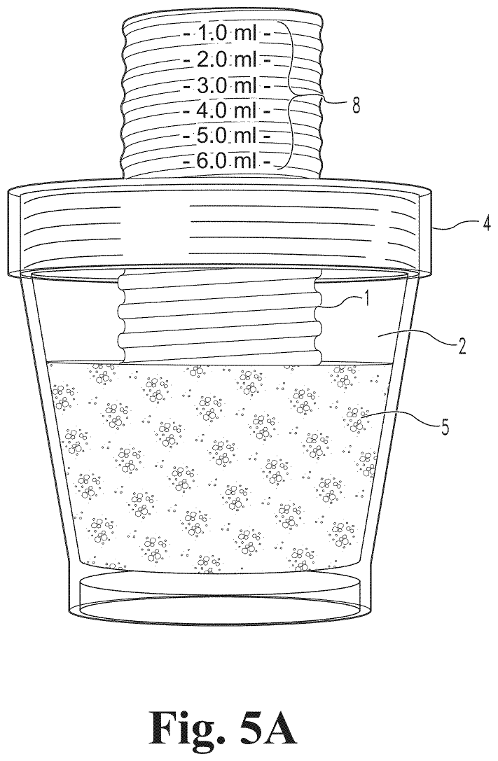

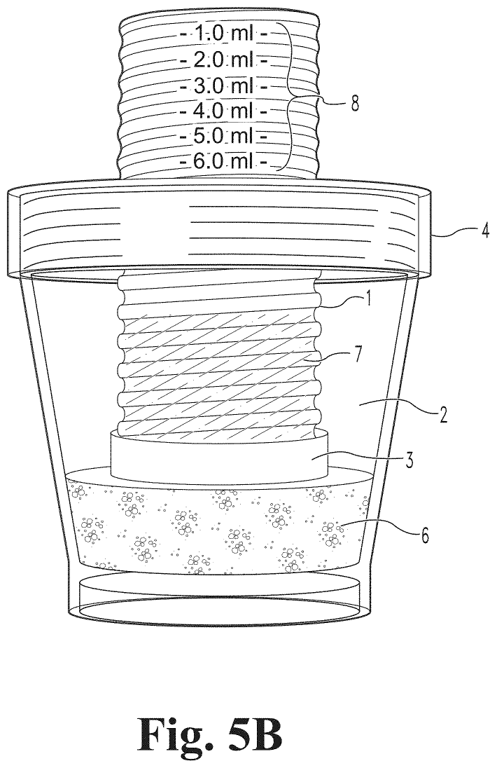

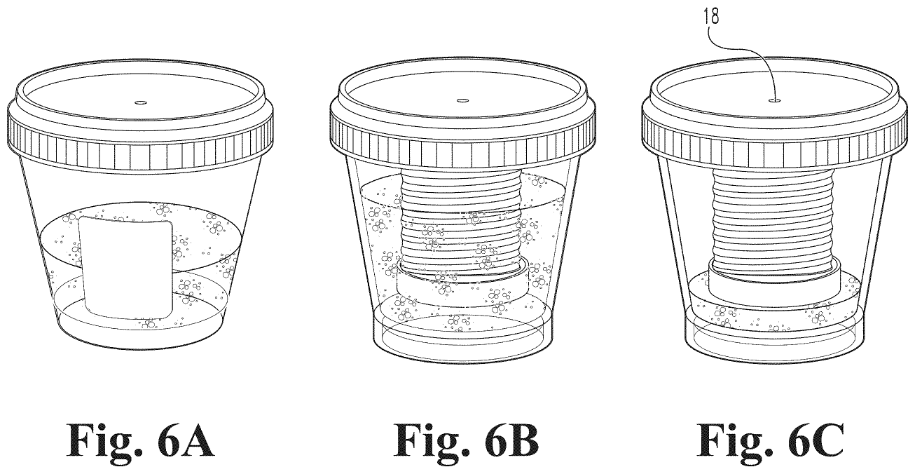

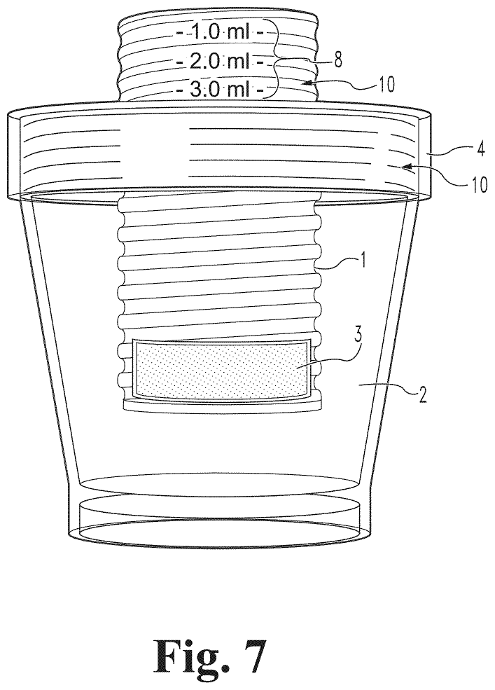

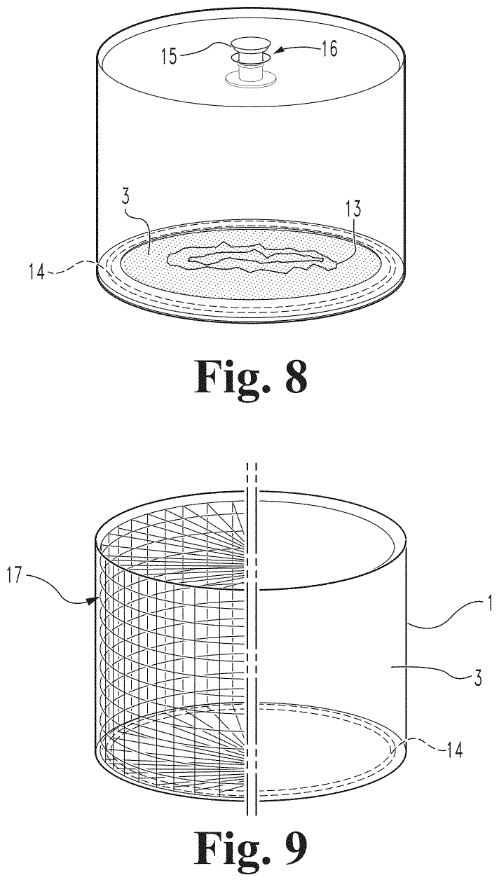

FIG. 1 illustrates a device suitable for the concentration of a sample having a relatively small volume. The active reservoir 1 includes a permeable membrane 3 covering the orifice at its terminus. The active reservoir has external threads sized to engage a threaded orifice within cap 4 to allow the active reservoir to be rotationally positioned directionally within cap 4. The threads 10 can be replaced with parallel bands and cavities that allow the active reservoir 1 to be moved through the cap 4 in increments by applying sufficient force on the active reservoir to move the active reservoir through different engaged positions. The active reservoir can similarly be positioned within the cap 4 by friction and/or a flange. Active reservoir 1 including membrane 3 and cap 4 fits into sample reservoir 2 providing a clearance about active reservoir 1 and sample reservoir 2. FIG. 2 illustrates a device having the same features, but suitable for the concentration of a sample having a larger volume. FIG. 3A illustrates the device of FIG. 1 including a sample to be concentrated 5, whereas FIG. 3B illustrates the device of FIG. 1 including a sample that has been concentrated 6. FIGS. 4A and 4B illustrate a device having the same features as FIGS. 3A and 3B, but suitable for the concentration of a sample having a larger volume. FIGS. 5A and 5B illustrate a device having markings on the upper region of the active reservoir 1 configured in a setting to provide a concentrated sample 6 illustrated in FIG. 5B having a volume of 6.0 mL. FIG. 6A illustrates a sample reservoir containing a sample for concentration. FIG. 6B illustrates a combination of an active reservoir inserted into a sample reservoir containing a sample for concentration where the level of concentration is not adjustable. FIG. 6C illustrates a combination of an active reservoir inserted into a sample reservoir illustrated in FIG. 6B where the sample concentration has been completed and the sample reservoir includes a concentrated sample. FIG. 7 illustrates a device including an orifice covered with a permeable membrane located on the side of the active reservoir. An orifice/membrane combination located on the side of the active reservoir can completely encircle the lower end of the active reservoir as a single unit or as multiple orifice/membrane units. FIG. 8 illustrates an active reservoir that can limit the predetermined volume of water removed from a sample by its own internal volume. The active reservoir of FIG. 8 includes a permeable membrane 3, a weighted ring 14 to orient the active reservoir in a sample reservoir, a water binding agent 13, and an orifice/valve combination 16/15 configured to close and limit the amount of water removed from the sample reservoir to provide a predetermined volume.

Illustrated in FIG. 9, the active reservoir 1 can also be constructed of a permeable membrane 3 having the shape of a bag, an envelope, or any other shape suitable for insertion into the sample reservoir. The predetermined volume of water removed with this active reservoir is controlled by limiting the internal volume of the reservoir, with allowance for any stretching of the permeable membrane. The predetermined volume can also be controlled by including the active reservoir constructed from a permeable membrane within a generally rigid caged structure having openwork and a predetermined volume to limit the expanded volume of the active reservoir constructed of the permeable membrane included therein. Such a caged structure can be constructed from any rigid or generally rigid material including a mesh netting subject to minimal stretching.

A device illustrated in the FIGS. 1-7 and 15-21 can be utilized to:

(a) concentrate a sample by placing a desired volume of a sample within the sample reservoir 2, or the optional sample reservoir 19, combination of 2/19,

(b) inserting the permeable membrane end of active reservoir 1 which includes a water-binding agent, into sample reservoir 2, 19, or 2/19, and

(c) engaging the cap 4. A stabilizer can be included in reservoir 2, 19, or 2/19 provided the material of interest in the sample would benefit from the stabilizer. If the device is fitted with markings that allow the concentration factor to be controlled, it is set once the sample is in place. Concentration begins immediately and continues until the level of the concentrate remaining in sample reservoir 2, 19, or 2/19 has receded to below the permeable membrane 3. If desired, when concentrate recedes below the permeable membrane the depth of the permeable membrane end of reservoir 1 can be further adjusted and the sample can be further concentrated.

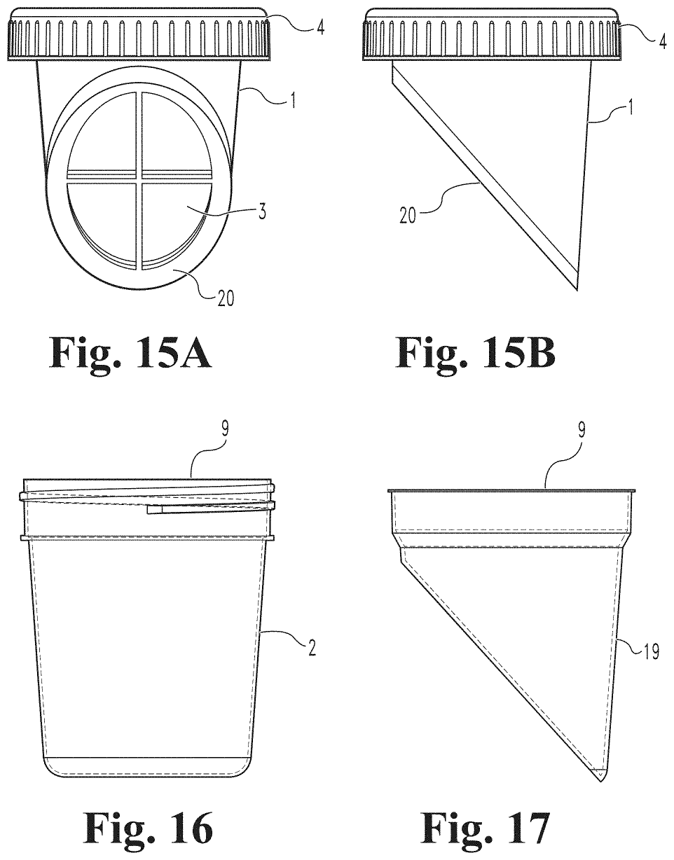

FIGS. 15A and 15B illustrate two views of the active reservoir 1 having a permeable membrane 3 covering the orifice 9 (not shown) and held in place with the membrane sealer 20. The active reservoir 1 illustrated in FIGS. 15A and 15B can be utilized for smaller samples by inserting the sample to be concentrated and active reservoir 1 (illustrated in FIGS. 15A and 15B) into the optional sample reservoir 19 (illustrated in FIG. 17). For stability, the combination of the active reservoir 1/optional sample reservoir 19 can be inserted into sample reservoir 2 illustrated in FIG. 16 through orifice 9. Once the sample has been introduced and the active reservoir 1 has been inserted into the sample reservoir (2, 19, or the combination of 2/19), and the cap tightened, the concentration can take place regardless of the orientation of the assembled device.

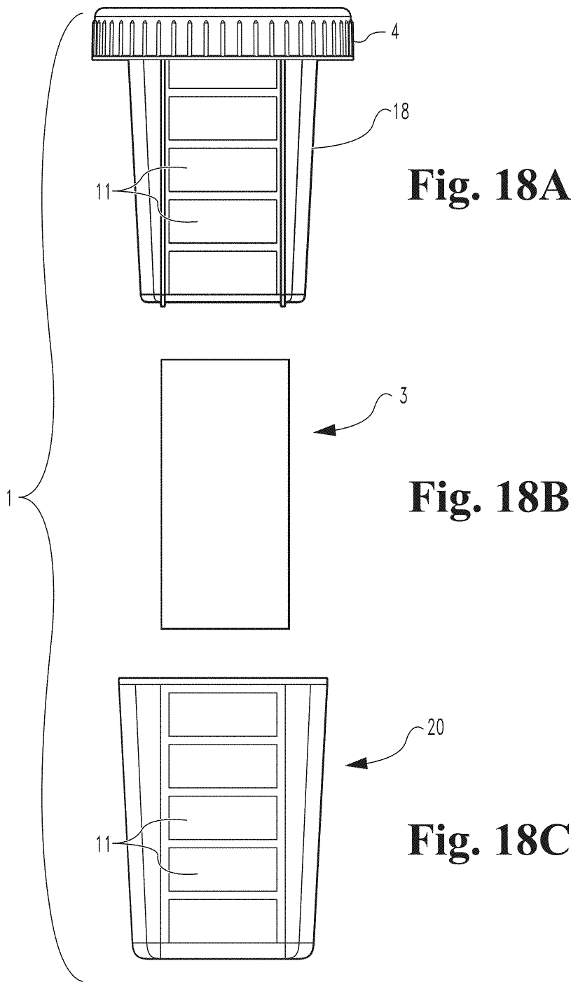

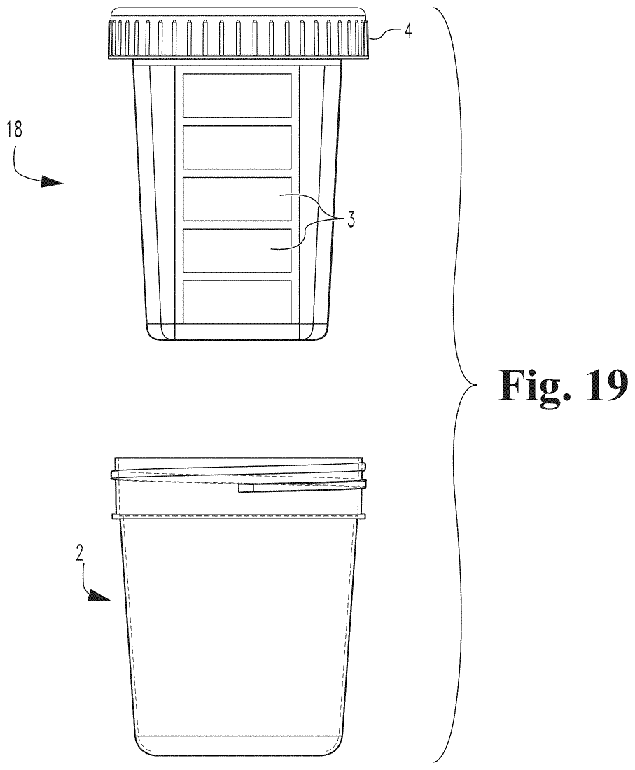

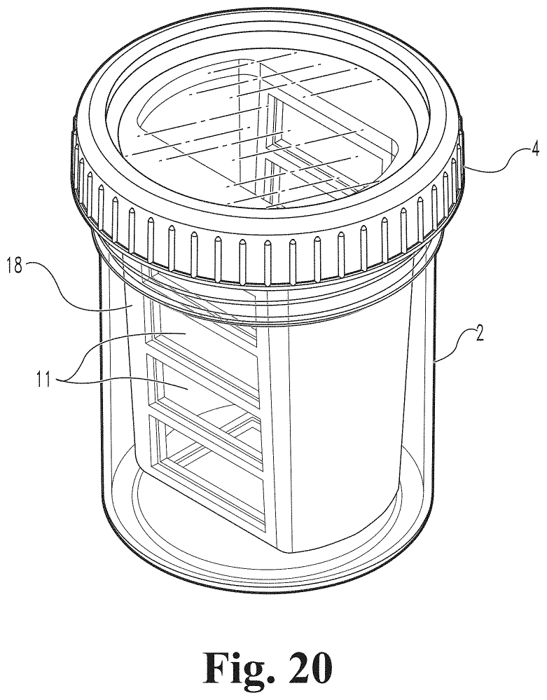

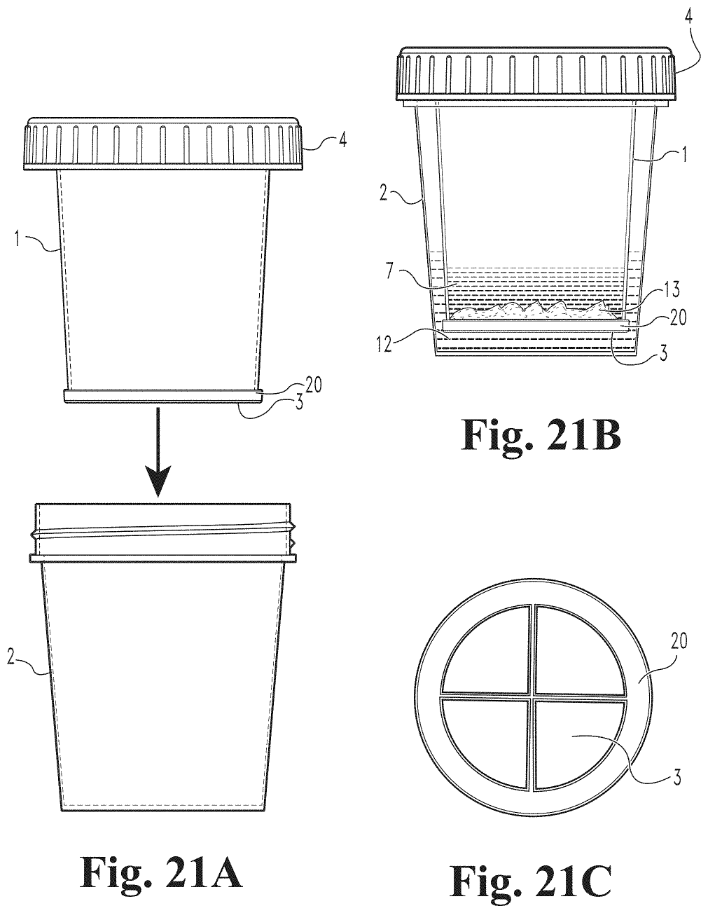

FIGS. 18A, 18B, and 18C illustrate frontal views of the components of an embodiment of active reservoir 1, having a plurality of orifices 11 on two outside walls and the bottom of the active reservoir 1 (illustrated in FIG. 18A). During assembly, the permeable membrane 3, illustrated in FIG. 18B, is placed over the plurality of orifices 11 and the combination inserted into the membrane sealer 20, illustrated in FIG. 18C, to fix the permeable membrane 3 over the plurality of orifices 11. Optionally, the orifices 11 can be covered by individual membranes (not shown) and similarly held in place with the membrane sealer 20. Fully assembled, the active reservoir 1 illustrated in FIG. 19, containing a water binding material 13 (illustrated in FIG. 21B) is inserted into the sample reservoir 2 containing a unconcentrated sample 5 (illustrated in FIG. 3A) to be concentrated. The increased number of orifice/membrane combinations along the sides and the bottom of the active reservoir 1 and the water binding material 13 contained therein facilitates the rapid concentration of the aqueous unconcentrated sample 5 included in the sample reservoir 2. FIG. 20 illustrates the active reservoir 1 illustrated in FIGS. 18A, 18B, and 18C (without placement of the permeable membrane) positioned in the sample reservoir 2 (illustrated in FIG. 19 to illustrate the orifices 11 positioned on the sides and the bottom of the active reservoir 1).

FIGS. 21A, 21B, and 21C illustrate a further embodiment where the active reservoir 1 includes a permeable membrane 3 held in place over orifice 9 (not shown) located on the bottom of the active reservoir 1 and held in place with the membrane sealer 20. Further, the active reservoir 1 in these FIGS. 21A, 21B, and 21C) further include a cap 4 attached to the active reservoir 1. FIG. 21C illustrates a suitable permeable membrane/membrane sealer (3/20) combination utilized in active reservoir 1 illustrated in FIG. 21A. The active reservoir 1 containing a water binding material 13 (illustrated in FIG. 21B) is configured to be inserted into the sample reservoir 2 containing a sample for concentration. FIG. 21B illustrates the active reservoir 1 containing the water binding material inserted into sample reservoir 2 in the process of concentrating a sample. The assembled device illustrated in FIG. 21B contains a partially concentrated sample 12 in the sample reservoir 2 and water removed in the active reservoir 7 along with the water binding material 13 positioned in the active reservoir 1. As with several previously discussed devices, the device illustrated in FIG. 21B can continue to concentrate a sample regardless of its orientation. Finally, once the concentration is complete, the active reservoir 1 can be removed and the sample reservoir covered with a new cap and transported for testing or could be tested directly within the sample reservoir 2.

Samples particularly suitable for concentration utilizing the disclosed device include, but are not limited to general solutes, biological fluids, and industrial fluids. Examples of general solutes include amino acids, polypeptides, antibodies, enzymes, cholesterols, lipids, carbohydrates, antigens, nucleic acid monomers, nucleic acid polymers, unicellular organisms, multicellular organisms, cells, viruses, bacteriophages, vitamins, cell signalling molecules, pulps, heavy metals, elements/chemicals, fertilizers, whey, glycols, acids, alcohols, bases, and dyes. Examples of biological liquids that can be concentrated in the disclosed device include, but are not limited to, blood, serum, plasma, breast milk, urine, saliva, sputum, BAL, amniotic fluid, bone marrow, synovial fluid, CSF, pleural fluid, peritoneal fluid, vaginal fluid, semen, mucus, stool, sweat, gastric fluid, pancreatic juice, tears, breath condensate, and vitreous fluid. Examples of industrial liquids that can be concentrated include, but are not limited to, fracking waste, food and beverage, chemical waste, pharmaceutical products/biproducts/waste, cooling water, suspended heavy metals, toxic compounds, brine, textile dyes, microalgae cultures, bacterial cultures, viral cultures, cell cultures, sewage, and environmental water.

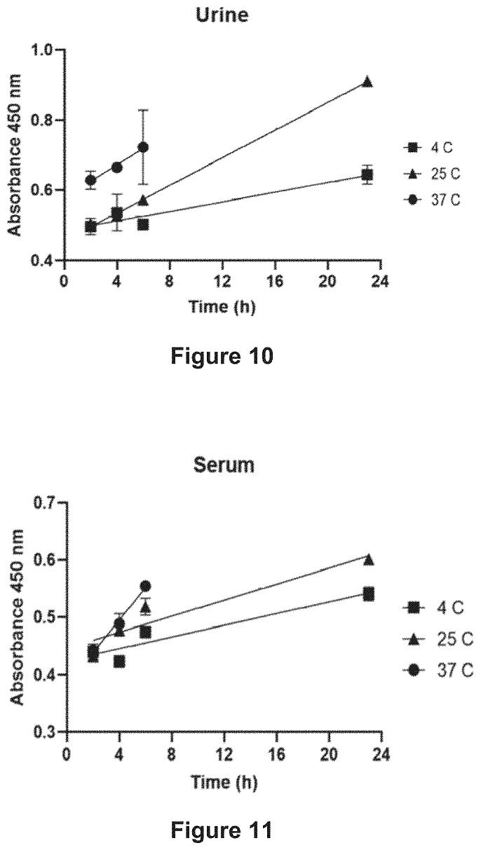

Sample concentrations have been successfully carried out at temperatures ranging from just above 0° C. to near 40° C. The flux rate was slower at the lower temperatures. Artificial urine and human serum were spiked with polyclonal antibodies raised against Shigella bacterial antigens. ELISA was performed using concentrated samples collected over time to quantify the increase in sensitivity following concentration for up to 24 h at three temperatures. A linear regression line was fit to the duplicate data with plotted averages and standard deviation of the mean. As demonstrated in FIGS. 10 and 11, both samples of urine and serum were concentrated. Concentration occurred faster at 37° C. than at 4° C.

Embodiments of the reservoirs for the device are constructed of polymeric materials, paper, or metals. The reservoir components of the device can be readily manufactured by methods that include, but are not limited to injection molding, 3-D printing, stamping, and the like. Examples of materials suitable for injection molding include, but are not limited to, ABS (Acrylonitrile Butadiene Styrene), PC (polycarbonate), PPA (Aliphatic Polyamides), POM (Polyoxymethylene), PMMA (Polymethyl Methacrylate), PP (Polypropylene), PBT (Polybutylene Terephthalate) and polyethylene (PE). Reservoirs made from clear polymeric materials that minimally absorb, adsorb or bind components from the samples are preferred.

Suitable permeable membranes generally include, but are not limited to:
Thin-film composite membranes (TFC) having a polyamide nonporous solute rejection layer on a porous nanocomposite support;
MWCO (Molecular Weight Cutoff) membrane;
Cellulose triacetate (CTA) on a woven mesh;
Hollow fiber membranes having modular structure with a lumen side and a shell side for feed and draw solutions, respectively.

Such membranes can generally be obtained from:
Fluid Technology Solutions, Inc.,
Aromatec,
Aquaporin,
Koch Separation Systems, Porifera, and
Toyobo

In the present work, the ultrafiltration and nanofiltration membranes were also modified by forming a polyamide selective layer on one side (the active side) of the membrane. The procedure utilized involves dispensing M-phenylenediamine (MPD) onto a nanofiltration membrane (NF) or an ultrafiltration membrane (UF), pouring off the MPD and immersing the membrane in a solution of trimesoyl chloride (TMC) in hexane, removing the membrane and drying the membrane at least overnight.

Methods for the attachment of the membrane over the first orifice of the active reservoir include, but are not limited to, thermal welding (hot gas, hot wedge, extrusion, hot plate, infrared, and laser), mechanical welding (spin, stir, vibration, and ultrasonic), electromagnetic welding (induction, dielectric, microwave, resistance/implant/electrofusion), adhesive/potting/epoxy/silicone, mechanical deformation (pressure with or without an O-ring seal) melted glass, and grease.

Suitable water-binding agents include any material capable of binding to and taking up water to facilitate passage of water across the permeable membrane. The binding of water to the agent can be through chemical reaction, adsorption, and/or absorption. Suitable water-binding materials include, but are not limited to hydrogels, super absorbent polymers, water absorbing desiccants, salts, solutes, and combinations thereof. Because of their high water-binding capacity, superabsorbent hydrogels have proven particularly effective as water-binding agents.

In order to prevent degradation and undesired changes in the sample between collection, concentration, and analysis, stabilizers can be added to the sample concentrated or being concentrated. The nature of the stabilizer is dependent on the nature of the sample being stabilized. The purpose of the stabilizer is to maintain the concentrated sample in the same or substantially the same condition with regard to materials being analyzed. Stabilization may require inactivation of one or more enzymes, the blocking of a decomposition reaction, maintaining a desired pH, and the like. Preferred stabilizers added to the sample prior to concentration achieve the desired outcome without adversely affecting the rate of concentration.

Preferred stabilizers protect the component of interest during concentration and don't interfere with the concentration process. Stabilizers that interfere with the concentration step can be added to the concentrate at the end of the process. Examples of suitable stabilizers are listed in Table 1.

TABLE 1

| | | Working |
|---|---|---|
| Compounds | Mode of action | concentration |
| Osmolyte stabilizers, polyols and sugars: | Stabilize the lattice structure of the water, | 10-40% |
| Glycerol, erythritol, arabitol, sorbitol, | increasing the surface tension and viscosity. | |
| mannitol, xylitol, mannisdomannitol, | Stabilize hydration shells and protect against | |
| glucosylglycerol, glucose, fructose, | aggregation by increasing the molecular | |
| sucrose, trehalose | density of the solution without changing the | |
| | dielectric constant. | |
| Polymers: | Polymers increase molecular density and | 1-15% |
| Dextran's, hydroxyl ethyl starch (HETA), | solvent viscosity, thus lowering protein | |
| gelatin, levans, polyethylene glycol, | aggregation in a single-phase system. | |
| polysaccharides, inert proteins | | |
| Amino acid and their derivatives: | Small amino acids with no net charge have | 20-500 mM |
| Glycine, alanine, proline, histidine, | weak electrostatic interactions with proteins. | |
| arginine, methionine, taurine, betaine, | Octopine is a derivative of arginine that is | |
| octopine, glutamate, sarcosine, Y | less denaturing to proteins. TMAO stabilizes | |
| aminobutyric acid, trimethylamine-oxide | proteins even in presence of denaturants | |
| (TMAO) | such as urea. Most of these compounds | |
| | increase the surface tension of water. | |
| Ionic stabilizers: | Larger anions shield charges and can | 20-400 mM |
| Salts, citrate, sulfates, acetate, boric acid, | stabilize proteins at low concentrations. At | |
| ammonium sulfate, phosphates, | high concentrations, they lead to | |
| quaternary amines | precipitation due to competition for water | |
| | molecules. | |
| Surfactants: | Suppress aggregation, nonspecific | 0.0003-0.03% |
| Polysorbate80, polysorbate 20, Brij35, | adsorption and assist in protein folding | |
| Triton X-10, Pluronic F127, sodium | | |
| dodecyl sulfate (SDS), biosurfactants | | |
| Preservatives: | Inhibit microbial growth, works better with | <1% |
| Benzyl-alcohol, m-cresol, phenol | co-elute like sucrose | |

Addition of a stabilizer may be optimal following concentration due to the high viscosity and concentration of dissolved solute in this stabilizer formulation. In some tests, when added prior to concentration, flux in the concentrator was lowered by a factor of 5. For stabilizers that substantially reduce the rate of concentration it is preferred that the stabilizer be added to the sample as a pre-measured powder following concentration and sample collection.

### Increased Sensitivity in Serological Assays:

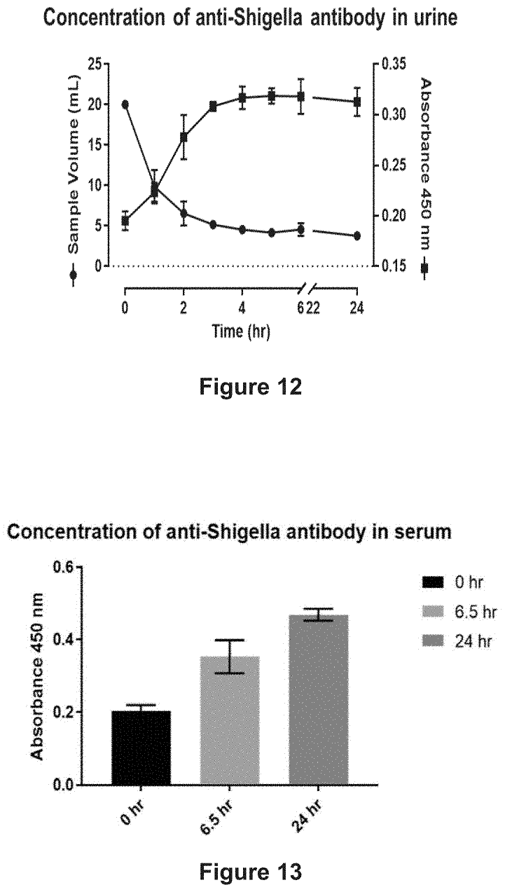

ELISA assays were used to demonstrate assay sensitivity increase following concentrations carried out in the device. First, a Shigella ELISA kit was used for detection in urine. Shigella antibody PA1-7245 was spiked at 1:10,000 into urine and the solution was concentrated for 24 h at 22° C. For the experiment, 14 devices were used and pairs of devices were devoted to each hourly timepoint. No stabilization additive was used. As illustrated in FIG. 12, the final volume of the devices after 24 h was 3.75 mL, a 5.3-fold decrease of volume. As volume decreased, the absorbance at 450 nm increased.

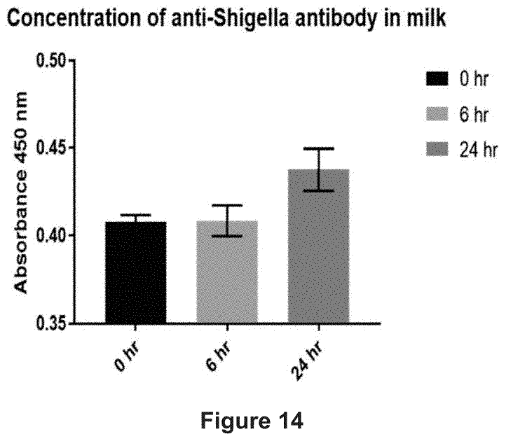

Similar experiments, illustrated in FIGS. 13 and 14, were performed using Shigella antibodies spiked into human serum and whole milk, demonstrating the ability to concentrate the samples and enhance the absorbance at 450 nm.

### Concentration of BSA Samples:

Samples of bovine serum albumin (BSA) in either artificial urine (AU) or deionized water (diH₂O) were prepared and concentrated for 90 minutes. Samples were analyzed using a Pierce 660 Assay following the manufacturer's instructions. Concentration of the BSA solutions were carried out with two different devices. The results shown below in Table 2 and 3 were obtained using the device shown in FIGS. 21A through 21C. The results shown below in Tables 4 were obtained using the device shown in FIGS. 15A, 15B, 16, and 17.

TABLE 2

| | | | |
|---|---|---|---|
| Media | AU | AU | AU |
| Starting Sample Volume (mL) | 20 | 20 | 20 |
| Analyte | BSA | BSA | BSA |
| Initial Concentration (μg/mL) | 54 | 54 | 54 |
| Concentration time (min) | 90 | 90 | 90 |
| Final Concentration (μg/mL) | 337 | 301 | 202 |
| Final Sample Volume (mL) | 2.0 | 2.3 | 3.2 |
| Concentration Factor (by volume) | 10X | 8.7X | 6.3X |
| Concentration Factor (by Pierce 660) | 6.2X | 5.6X | 3.7X |

TABLE 3

| | | |
|---|---|---|
| Media | diH2O | diH2O |
| Starting Sample Volume (mL) | 20 | 20 |
| Analyte | BSA | BSA |
| Initial concentration (μg/mL) | 21.2 | 21.2 |
| Concentration time (min) | 90 | 90 |
| Final concentration (μg/mL) | 573 | 555 |
| Final Sample Volume (mL) | 0.94 | 1.0 |
| Concentration Factor (by volume) | 21.3X | 20X |
| Concentration Factor (by Pierce 660) | 27.0X | 26.2X |

TABLE 4

| | | |
|---|---|---|
| Media | AU | AU |
| Starting Sample Volume (mL) | 10 | 10 |
| Analyte | BSA | BSA |
| Initial concentration (μg/mL) | 60 | 60 |
| Concentration time (min) | 90 | 90 |
| Final concentration (μg/mL) | 192 | 204 |
| Final Sample Volume (mL) | 1.5 | 1.8 |
| Concentration Factor (by volume) | 6.7X | 5.6X |
| Concentration Factor (by Pierce 660) | 3.2X | 3.4X |

### Concentration of COVID Antigen:

The buffer (0.0125% sodium azide in sterile, nanopure water) provided with a commercially available COVID-19 Rapid, At Home Lateral Flow Assay (LFA) was spiked with COVID-19 recombinant spike protein at a concentration below reported limits of detection. A negative test result was confirmed by running the unconcentrated sample on the same commercially available COVID-19 Rapid LFA kit following the manufacturer's instructions. The sample was then concentrated by a factor of 12 using the device illustrated in FIGS. 15A, 15B, 16, and 17 for 90 min. The 12× concentrated sample was analyzed on the same manufacturer's LFAs according to the same procedure and tested positive. The results and conditions are outlined in Table 5.

TABLE 5

| | | |
|---|---|---|
| Media | LFA Buffer | LFA Buffer |
| Starting Sample Volume (mL) | 10 | 10 |
| Analyte | COVID-19 | COVID-19 |
| | Spike | Spike |
| Initial Concentration (ng/mL) | 2.5 | 2.5 |
| Concentration time (min) | 90 | 90 |
| Final Sample Volume (mL) | 0.85 | 0.85 |
| Concentration Factor (by volume) | 12X | 12X |
| Pre-concentration LFA result (+/−) | − | − |
| Post-concentration LFA result (+/−) | + | + |

### Concentration of Glucose Samples:

Samples (20 mL and 10 mL) of artificial urine (AU) were spiked with glucose and the solutions were concentrated for 90 min. The 20 mL samples were concentrated using the device illustrated in FIGS. 21A through 21C, and the results are shown in Table 6. The results of concentrating 10 mL of glucose/AU solution are shown in Table 7 using the device illustrated in FIGS. 15A, 15B, 16, and 17. The glucose concentrations were determined by Pierce 660 Assay following the manufacturer's instructions.

TABLE 6

| | | | |
|---|---|---|---|
| Media | AU | AU | AU |
| Starting Sample Volume (mL) | 20 | 20 | 20 |
| Analyte | Glucose | Glucose | Glucose |
| Initial concentration (mg/mL) | 0.63 | 0.63 | 0.63 |
| Concentration time (min) | 90 | 90 | 90 |
| Final concentration (mg/mL) | 2.43 | 2.08 | 2.51 |
| Final Sample Volume (mL) | 2.9 | 3.4 | 2.8 |
| Concentration Factor (by volume) | 6.9X | 5.9X | 7.1X |
| Concentration Factor (by Pierce 660) | 3.9X | 3.3X | 4.0X |

TABLE 7

| | | |
|---|---|---|
| Media | AU | AU |
| Starting Sample Volume (mL) | 10 | 10 |
| Analyte | Glucose | Glucose |
| Initial concentration (mg/mL) | 0.63 | 0.63 |
| Concentration time (min) | 90 | 90 |
| Final concentration (mg/mL) | 2.26 | 1.49 |
| Final Sample Volume (mL) | 1.4 | 2.0 |
| Concentration Factor (by volume) | 7.1X | 5.0X |
| Concentration Factor (by Pierce 660) | 3.6X | 2.4X |

### Concentration of Chemical Weapon Simulants:

Two solutions of mustard simulants, 2-chloroethyl ethyl sulfide (CEES) and 2-chloroethyl phenyl sulfide (CEPS) were prepared in deionized water (diH2O). The prepared solutions included CEES and CEPS at concentrations below the detection limits using M8 or M9 paper. The dilute solutions were concentrated for 90 min. The starting concentration of CEES was 1.25 ng/mL and was not detectable using M8 or M9 paper. The starting concentration of CEPS was 5 ng/mL and this was not detectable using M8 or M9 paper. After 90 minutes of concentration, CEES and CEPS were applied to new sheets of M8 and M9 paper and both indicated a positive result. These results are shown in Table 8 and concentration occurred using the apparatus shown in FIGS. 21A through 21C.

TABLE 8

| | | |
|---|---|---|
| Media | diH₂O | diH₂O |
| Starting Sample Volume (mL) | 20 | 20 |
| Analyte | CEES | CEPS |
| Initial concentration (ng/mL) | 1.25 | 5 |
| Initial Test Result (M8 paper, +/−) | − | − |
| Initial Test Result (M9 paper, +/−) | − | − |
| Concentration time (min) | 90 | 90 |
| Final Sample Volume (mL) | 2 | 2 |
| Final Result (M8 paper, +/−) | + | + |
| Final Result (M9 paper, +/−) | + | + |

While applicant's disclosure has been provided with reference to specific embodiments above, it will be understood that modifications and alterations in the embodiments disclosed may be made by those practiced in the art without departing from the spirit and scope of the invention. All such modifications and alterations are intended to be covered.

### ELEMENTS INCLUDED IN THE FIGURES

1 active reservoir
2 sample reservoir
3 permeable membrane
4 cap
5 unconcentrated sample
6 concentrated sample
7 water removed
8 markings
9 orifice
10 threads
11 multiple orifices
12 partially concentrated sample
13 water binding material
14 weighted ring
15 valve
16 valve orifice
17 cage structure
18 active reservoir component
19 optional sample reservoir
20 membrane sealer

1. A device for concentrating liquids comprising,
a sample reservoir and an active reservoir,
wherein the active reservoir includes at least one orifice and a permeable membrane covering the at least one orifice and contains at least one water binding material therein,
wherein the sample reservoir includes at least one orifice and is configured to receive an aqueous sample, to removably receive the active reservoir therein, and to provide contact between the aqueous sample and the permeable membrane,
wherein the active reservoir is configured to receive, through the permeable membrane, an aqueous portion of the sample from the sample reservoir, retain at least some of the aqueous portion received by the water binding material, and provide a concentrated sample in the sample reservoir.

2. The device of claim 1, wherein the active reservoir includes a plurality of orifices and a permeable membrane covering each of the plurality of orifices.

3. The device of claim 1, wherein the device is configured to operate to provide a concentrated sample regardless of its orientation.

4. The device of claim 1, wherein the device includes an adjusting mechanism in communication with the active and sample reservoirs and is configured to control the insertion depth of the active reservoir.

5. The device of claim 1, wherein the sample reservoir utilized includes an optional sample reservoir having a reduced volume.

6. The device of claim 1, wherein the active reservoir includes a water-binding material selected from the group consisting of a hydrogel, a super absorbent polymer, water absorbing desiccant, a salt, a solute, and combinations thereof.

7. The device of claim 1, wherein the sample reservoir includes a preservative material configured to preserve and stabilize biological and/or chemical materials added thereto.

8. The device of claim 7, wherein the preservative material is selected from the group consisting of osmolytes, polyols, sugars, polymers, amino acids, anionic compounds, cationic compounds, surfactants, biocides, and derivatives of thereof.

9. A method for concentrating an aqueous sample, utilizing the device of claim 1, comprising:
(a) adding a sample to the sample reservoir of the device of claim 1;
(b) assembling the device of claim 1, wherein assembling the device of claim 1 provides sample contact with the membrane of the active reservoir;
(c) allowing the sample to equilibrate between the active and sample reservoirs until a desired measure of concentration has been achieved.

10. The method of claim 9, wherein the aqueous sample is a biological liquid.

11. The method of claim 10, wherein the biological liquid is selected from the group consisting of blood, serum, plasma, breast milk, urine, saliva, sputum, BAL, amniotic fluid, bone marrow, synovial fluid, cerebrospinal fluid, pleural fluid, peritoneal fluid, vaginal fluid, semen, mucus, stool, sweat, gastric fluid, pancreatic juice, tears, breath condensate, and vitreous fluid.

12. The method of claim 9, wherein the aqueous sample is an environmental or industrial fluid selected from the group consisting of natural water, wastewater, produced water, rain-water, and agricultural runoff.

13. The method of claim 9, wherein the sample selected contains a general solute.

14. The method of claim 13, wherein the general solute is selected from the group consisting of amino acids, polypeptides, antibodies, enzymes, cholesterols, lipids, carbohydrates, antigens, nucleic acid monomers, nucleic acid polymers, unicellular organisms, multicellular organisms, cells, viruses, bacteriophages, vitamins, cell signaling molecules, pulps, heavy metals, elements/chemicals, fertilizers, wheys, glycols, acids, alcohols, bases, and dyes.

15. The method of claim 9, further comprising adding a preservative material to the sample reservoir, wherein the material is configured to preserve and stabilize any samples added thereto.

16. The method of claim 15, wherein the sample is a biological sample and the preservative material added thereto is selected from the group consisting of osmolytes, polyols, sugars, polymers, amino acids, anionic compounds, cationic compounds, surfactants, biocides, and derivatives of thereof.

17. The method of claim 9, wherein allowing the liquid to equilibrate between the active and sample reservoirs until the desired measure of concentration has been achieved involves allowing the liquid to equilibrate for at least about 5 minutes producing a sample concentrate.

18. The method of claim 17, wherein after equilibration, the active reservoir is removed from the sample reservoir containing the sample concentrate, the sample reservoir is capped, and the capped sample reservoir is labeled for storage or transportation until analysis can take place.

19. The method of claim 9, wherein a portion of sample concentrate is removed for testing.

20. A device for concentrating liquids comprising,
a sample reservoir and an active reservoir,
wherein the sample reservoir is configured to receive an aqueous sample for concentration and to receive the active reservoir therein,
wherein the active reservoir is constructed of a permeable membrane having a water binding material therein, and a predetermined volume that is configured to control a final volume of an aqueous portion of the sample to be contained in the sample reservoir when the active reservoir is filled and expanded to a maximum volume.

21. A device for concentrating liquids comprising,
a sample reservoir and an active reservoir,
wherein the sample reservoir is configured to receive an aqueous sample for concentration and to receive the active reservoir therein,
wherein the active reservoir is constructed of a permeable membrane having a water binding material therein,
wherein the active reservoir is included within a rigid caged structure having openwork and a predetermined volume configured to limit the final volume of an aqueous portion of the sample to be contained in the sample reservoir when the active reservoir is filled and expanded to a maximum volume.

22. A device for concentrating liquids comprising a sample reservoir and an active reservoir,
wherein the sample and active reservoirs are configured to provide passage of water into the active reservoir through a permeable membrane,
wherein the active reservoir includes a water binding material, and
wherein the sample reservoir is configured to:
contain an aqueous sample in contact with the permeable membrane,
provide passage of water through the permeable membrane into the active reservoir,
cause the water to bind to the water binding material and
provide a concentrated sample in the sample reservoir.

23. A kit for constructing a device for concentrating an aqueous sample, the kit comprising and active reservoir and a sample reservoir, wherein:
the active reservoir includes at least one orifice and a permeable membrane covering the at least one orifice on at least one surface and configured to be removably inserted into the sample reservoir;
the sample reservoir is configured to receive (a) an aqueous sample for concentration, and (b) a sufficient portion of the active reservoir to provide contact with the aqueous sample included therein; and
a water binding material in or for addition to the active reservoir.

24. The kit of claim 23, wherein the kit further includes at least one preservative.
